(19) United States
(12) Patent Application Publication
(10) Pub. No.: US 2014/0080791 A1
(43) Pub. Date: Mar. 20, 2014

(54) OMEGA-3 PHOSPHOLIPID SUPPLEMENTS FOR FEMALES

(71) Applicant: Aker Biomarine AS, Oslo (NO)

(72) Inventors: Kjetil Berge, Oslo (NO); Nils Hoem, Oslo (NO)

(21) Appl. No.: 14/028,738

(22) Filed: Sep. 17, 2013

Related U.S. Application Data
(60) Provisional application No. 61/703,009, filed on Sep. 19, 2012.

(51) Int. Cl. A61K 31/661

(52) U.S. Cl. CPC A61K 31/661
USPC 514/120

(57) ABSTRACT

The invention relates to omega-3 phospholipid supplements for females, and in particular to omega-3 phospholipid supplements with increased bioavailability as evidenced by increased EPA and DHA in plasma phospholipids and an increase in omega-3 index as compared to males. In preferred embodiments, the omega-3 phospholipid is krill oil.

### CROSS-REFERENCE TO RELATED APPLICATION

The present application claims priority to pending U.S. Provisional Patent Application No. 61/703,009, filed Sep. 19, 2012, the contents of which are incorporated by reference in its entirety.

### FIELD OF THE INVENTION

The invention relates to omega-3 phospholipid supplements for females, and in particular to omega-3 phospholipid supplements with increased bioavailability as evidenced by increased EPA and DHA in plasma phospholipids and an increase in omega-3 index as compared to males.

### BACKGROUND OF THE INVENTION

Krill is a small crustacean which lives in all the major oceans world-wide. For example, it can be found in the Pacific Ocean (Euphausia pacifica), in the Northern Atlantic (Meganyctiphanes norvegica) and in the Southern Ocean off the coast of Antarctica (Euphausia superba). Krill is a key species in the ocean as it is the food source for many animals such as fish, birds, sharks and whales. Krill can be found in large quantities in the ocean and the total biomass of Antarctic krill (E. superba) is estimated to be in the range of 300-500 million metric tons. Antarctic krill feeds on phytoplankton during the short Antarctic summer During winter, however, its food supply is limited to ice algae, bacteria, marine detritus as well as depleting body protein for energy. Virtue et al., Mar. Biol. (1996) 126, 521-527. For this reason, the nutritional values of krill vary during the season and to some extent annually. Phleger et al., Comp. Biochem. Physiol. 131B (2002) 733.

The long-chain omega-3 polyunsaturated fatty acids DHA and EPA are popularly called omega-3. Supplementary intake of omega-3 is recommended in the western world, due to generally low dietary intake and omega-3′s health-promoting benefits. Benefits attributed to omega-3 include reduced risk and improved treatment outcomes regarding cardiovascular disease and inflammatory joint diseases. Better brain and central nervous system development, improved cognitive functioning, and improved skin health are additional benefits. Research indicates that even more omega-3 benefits for individuals will be identified and that greater intake can lead to better general health in western, industrialized cultures.

The omega-3 in krill oil is mainly in the omega-3 phospholipid form, which research suggests is a preferred dietary supplement when compared to omega-3 in triglyceride form. Marine omega-3 in dietary supplements is mostly derived from fish, such as fish body oil and cod liver oil, which provide omega-3 in triglyceride form. The omega-3 obtained from eating fatty fish such as salmon also provides some omega-3 in the phospholipid form.

### SUMMARY OF THE INVENTION

The invention relates to omega-3 phospholipid supplements for females, and in particular to omega-3 phospholipid supplements with increased bioavailability as evidenced by increased EPA and DHA in plasma phospholipids and an increase in omega-3 index as compared to males.

Accordingly, in some embodiments, the present invention provides methods for increasing the omega-3 phospholipid content of plasma phospholipids in a female subject as compared to male subjects comprising: administering an omega-3 phospholipid supplement to said female subject under conditions such that the omega-3 phospholipid content of plasma phospholipids in said female subject is increased. In some embodiments, the methods further comprise administering to the female subject from about 2 to 6 grams of said omega-3 phospholipid supplement for a period at least six weeks to effect an increase in omega-3 index of from about 1.8 to 2.5 fold, preferably about 2.2 fold, as compared to control subjects not receiving the treatment. In some embodiments, the methods further comprise administering to the female subject from about 2 to 6 grams of said omega-3 phospholipid supplement for a period at least twelve weeks to effect an increase in omega-3 index of from about 2.5 to 3.5 fold, preferably about 3.1 fold as compared to control subjects not receiving the treatment. In some embodiments, the female subject is not receiving a concurrent lipid altering therapy. In some embodiments, the administration effects a 35% to 55% increase in EPA in plasma phospholipids as compared to males and a 30% to 50% increase in DHA in plasma phospholipids as compared to males. In some embodiments, the administration effects a 20% to 40% increase in EPA in plasma phospholipids as compared to females receiving fish oil and a 30% to 50% increase in DHA in plasma phospholipids as compared to females receiving fish oil. As used above, the term “about” indicates a value of +/−5% of the stated value.

In some embodiments, the omega-3 phospholipid supplement is a krill oil, fish oil, fish roe oil, or fish byproduct oil. In some embodiments, the krill oil comprises from about 35% to 60%; from about 20% to 45% triglycerides on a w/w basis; and from about 50 to about 2500 mg/kg astaxanthin. In some embodiments, the composition comprises from about 3% to 10% ether phospholipids on a w/w basis, so that the total amount of ether phospholipids and non-ether phospholipids in the composition is from about 48% to 60% on a w/w basis. In some embodiments, the composition comprises from about 25% to 30% omega-3 fatty acids as a percentage of total fatty acids and wherein from about 80% to 90% of said omega-3 fatty acids are attached to said phospholipids. In some embodiments, the composition comprises from about 100 to about 2500 mg/kg astaxanthin. In some embodiments, the omega-3 supplement comprises from about 1% to about 10% w/w ether phospholipids; from about 27% to 50% w/w non-ether phospholipids so that the amount of total phospholipids in the composition is from about 30% to 60% w/w; from about 20% to 50% w/w triglycerides; from about 100 to about 2500 mg/kg astaxanthin; and from about 20% to 35% omega-3 fatty acids as a percentage of total fatty acids in said composition, wherein from about 70% to 95% of said omega-3 fatty acids are attached to said phospholipids. In some embodiments, the omega-3 is selected from EPA and DHA and combinations thereof In some embodiments, the female subject is a human. In some embodiments, the administration is oral.

In some embodiments, the present invention provides methods for increasing the omega-3 index in a female subject as compared to male subjects comprising: administering an omega-3 phospholipid supplement to said female subject under conditions such that omega-3 index in said female subject is increased. In some embodiments, the omega-3 phospholipid supplement is a krill oil. In some embodiments, the krill oil comprises from about 35% to 60%; from about 20% to 45% triglycerides on a w/w basis; and from about 50 to about 2500 mg/kg astaxanthin. In some embodiments, the composition comprises from about 3% to 10% ether phospholipids on a w/w basis, so that the total amount of ether phospholipids and non-ether phospholipids in the composition is from about 48% to 60% on a w/w basis. In some embodiments, the composition comprises from about 25% to 30% omega-3 fatty acids as a percentage of total fatty acids and wherein from about 80% to 90% of said omega-3 fatty acids are attached to said phospholipids. In some embodiments, the composition comprises from about 100 to about 2500 mg/kg astaxanthin. In some embodiments, the omega-3 supplement comprises from about 1% to about 10% w/w ether phospholipids; from about 27% to 50% w/w non-ether phospholipids so that the amount of total phospholipids in the composition is from about 30% to 60% w/w; from about 20% to 50% w/w triglycerides; from about 100 to about 2500 mg/kg astaxanthin; and from about 20% to 35% omega-3 fatty acids as a percentage of total fatty acids in said composition, wherein from about 70% to 95% of said omega-3 fatty acids are attached to said phospholipids. In some embodiments, the omega-3 is selected from EPA and DHA and combinations thereof In some embodiments, the female subject is a human. In some embodiments, the administration is oral.

In some embodiments, the present invention provides for use of an omega-3 phospholipid supplement to preferentially increase the omega-3 phospholipid content of plasma in a female subject.

In some embodiments, the present invention provides for use of an omega-3 phospholipid supplement to preferentially increase the omega-3 index in a female subject.

### DESCRIPTION OF THE FIGURES

FIG. 1 is graph comparing EPA in plasma phospholipids in males and females provided with krill oil or fish oil.

FIG. 2 is graph comparing DHA in plasma phospholipids in males and females provided with krill oil or fish oil.

FIG. 3 is graph comparing EPA and DHA in plasma phospholipids in males and females provided with krill oil.

FIG. 4 is graph comparing omega-3 index in males and females provided with krill oil or fish oil.

FIG. 5 is graph comparing omega-3 index in males and females provided with krill oil.

FIG. 6 is a graph comparing tolerability of fish and krill oil in males.

FIG. 7 is a graph comparing tolerability of fish and krill oil in females.

FIG. 8 is graph showing change in omega-3 index following 42 and 84 days of treatment with krill oil in males and females.

### DEFINITIONS

As used herein, “phospholipid” refers to an organic compound having the following general structure:

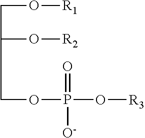

wherein R1 is a fatty acid residue, R2 is a fatty acid residue or —H, and R3 is a —H or a phospholipid headgroup moiety such as a choline (HOCH₂CH₂N⁺(CH₃)₃OH⁻) moiety, ethanolamine (HOCH₂CH₂NH₂) moiety, serine moiety, inositol moiety such as cyclohexane polyol inositol, and derivatives thereof. Preferably, R1 and R2 cannot simultaneously be —H. When R3 is an —H, the compound is a diacylglycerophosphate, while when R3 is a nitrogen-containing compound, the compound is a phosphatide such as lecithin, cephalin, phosphatidyl serine or plasmalogen.

An “ether phospholipid” as used herein refers to a phospholipid having an ether bond at position 1 of the glycerol backbone. Examples of ether phospholipids include, but are not limited to, alkylacylphosphatidylcholine (AAPC), lyso-alkylacylphosphatidylcholine (LAAPC), and alkylacylphosphatidylethanolamine (AAPE). A “non-ether phospholipid” is a phospholipid that does not have an ether bond at position 1 of the glycerol backbone.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term “omega-3 phospholipid” refers to phospholipids that at either the R1 and/or R2 positions comprise polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term “omega-3 phospholipid supplement” refers to a composition comprising natural or synthetic omega-3 phospholipids.

As used herein, astaxanthin refers to the following chemical structure:

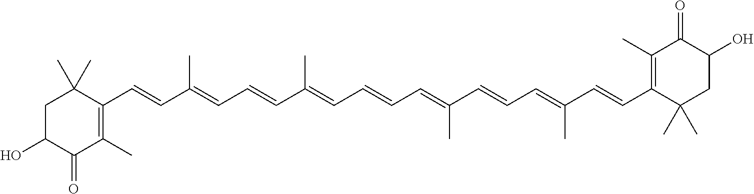 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

As used herein, astaxanthin esters refer to an astaxanthin molecule where a fatty acid is esterified to one or two of the OH groups in the molecule.

As used herein, the term w/w (weight/weight) refers to the amount of a given substance in a composition on weight basis. For example, a composition comprising 50% w/w phospholipids means that the mass of the phospholipids is 50% of the total mass of the composition (i.e., 50 grams of phospholipids in 100 grams of the composition, such as an oil).

As used herein, the term “fresh krill” refers to krill that is has been harvested less than about 12, 6, 4, 2 or preferably 1 hour prior to processing. “Fresh krill” is characterized in that products made from the fresh krill such as coagulum comprise less than 1 mg/100 g TMA, volatile nitrogen or Trimetylamine oxide-N, alone or in combination, and less than 1 g/100 g lysophosphatidylcholine.

As used herein the term “omega-3 index” is defined as the sum of EPA and DHA in erythrocyte membranes and is expressed as a percentage of total erythrocyte fatty acids. Harris W., Am J Clin Nutr (2008) 87(6); 1997S-2002S.

### DETAILED DESCRIPTION OF THE INVENTION

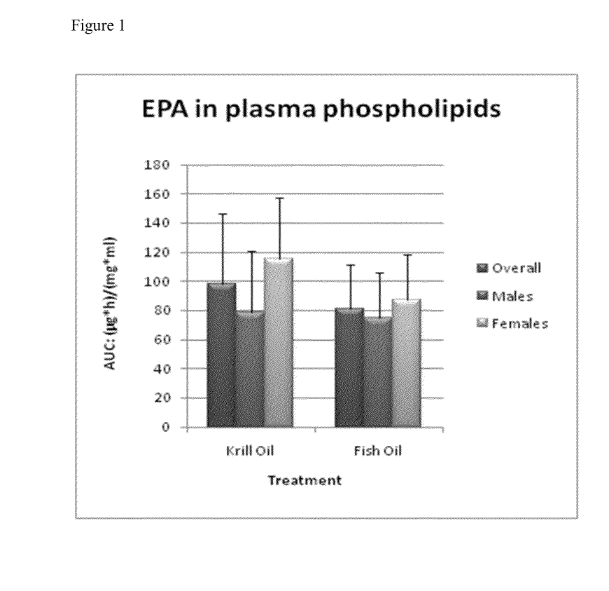

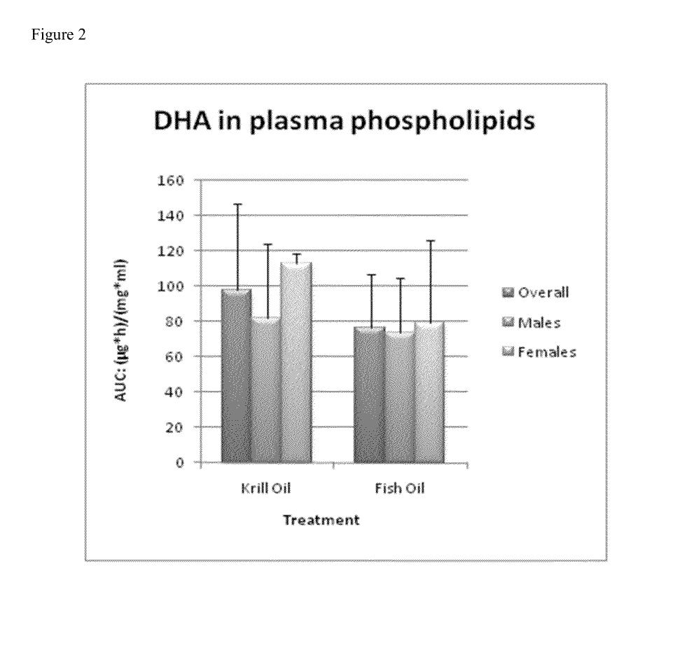

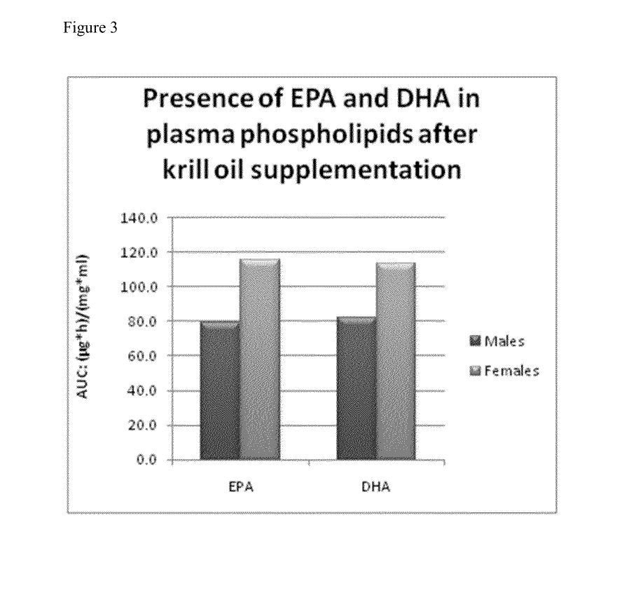

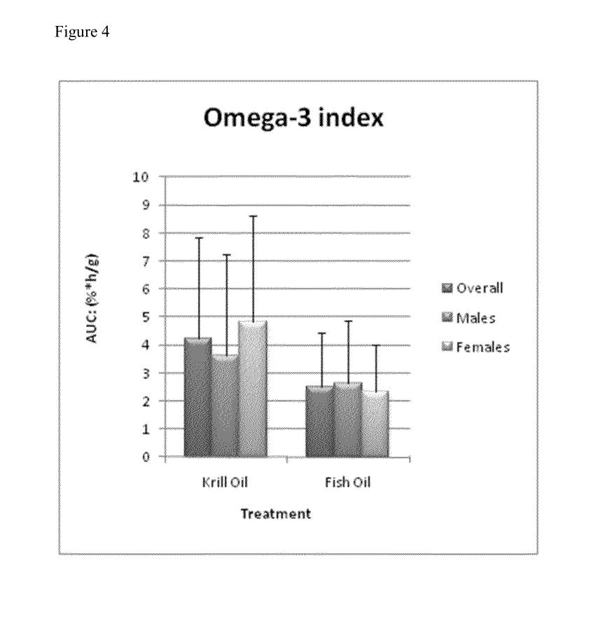

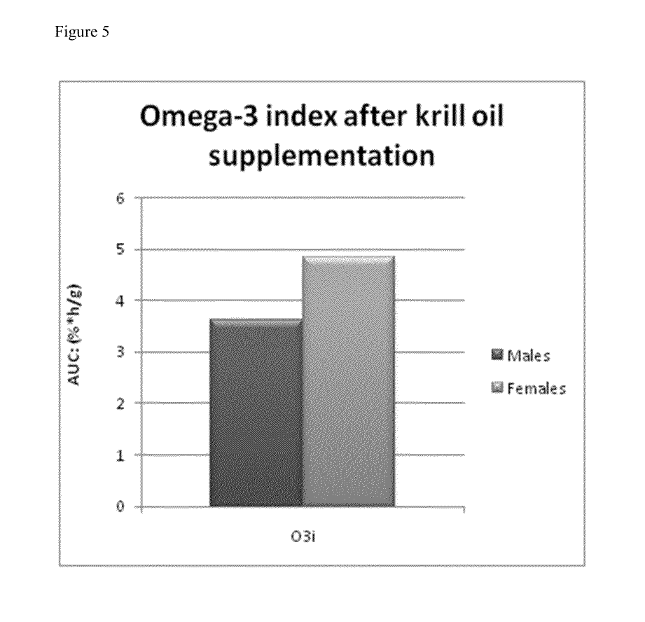

The invention relates to omega-3 phospholipid supplements for females, and in particular to omega-3 phospholipid supplements with increased bioavailability as evidenced by increased EPA and DHA in plasma phospholipids and an increase in omega-3 index as compared to males. The present invention provides for the use of the foregoing omega-3 phospholipid supplements in female subjects. Surprisingly, it has been found that there is distinct difference in the bioavailability of omega-3 phospholipids in male and female subjects. Female subjects provided with omega-3 phospholipid supplements exhibited a preferential increase in EPA and DHA content in plasma phospholipids as compared to male and also exhibited increased EPA and DHA content in plasma phospholipids as compared to subjects receiving fish oil. This data is summarized in FIGS. 1-8. Women provided with an omega-3 phospholipid supplement exhibited a 46% increase in EPA in plasma phospholipids as compared to males, a 38% increase in DHA in plasma phospholipids as compared to males, and a 33% higher increase in omega-3 index as compared to males. When compared with women receiving fish oil supplements, women receiving omega-3 phospholipid supplements exhibited 32% higher increase in EPA in plasma phospholipids, a 42% higher increase in DHA in plasma phospholipids, and a 106% higher increase in omega-3 index. Women receiving 4 grams krill oil demonstrated a 29% increase in omega-3 index after 42 days of treatment as compared to males and a 31% increase in omega-3 index after 42 days of treatment as compared to males. In some embodiments, the women demonstrated an increase of from 1.8 to 2.5 fold after 42 days of treatment and from about 2.5 to 3.5 fold following 84 days of treatment. These novel finding have significant implications for improving the cardiovascular health of women, as well as reducing inflammation (i.e. reducing the levels of TNF-α, IL-1 beta, IL-6, IL-10, TGF beta and fibrinogen in the blood) and the effects of metabolic syndrome in women. These effects include obesity, type-II diabetes, adipose tissue disfunction, fatty liver and heart, insulin resistance, high blood pressure, high cholesterol, high blood triglycerides, etc.

In preferred embodiments, the methods of the present invention utilize omega-3 phospholipids to increase DHA and/or EPA in the plasma lipids of females as compared to males and/or fish oil, or increase the omega-3 index in females as compared to males and/or fish oil. The omega-3 phospholipids may be naturally occurring, such as those obtained from krill (i.e., krill oil) or synthetic, such as those made by an enzymatic process. Suitable processes for synthetic omega-3 phospholipids are described in WO06/054183, WO02090560, WO05/037848, and WO05/038037, all of which are incorporated herein by reference. Suitable processes for producing krill oil include extraction with polar solvents such as ethanol, supercritical fluid extraction, extraction with non-polar organic solvents such as acetone, cold pressing, etc. See, e.g., WO2009/027692, WO2008/117062, WO2003/011873, all of which are incorporated herein by reference. In some embodiments, krill oil is extracted from the denatured krill meal. In some embodiments, the krill oil is extracted by contacting the krill meal with ethanol. In some embodiments, krill is then extracted with a ketone solvent such as acetone. In other embodiments, the krill oil is extracted by one or two step supercritical fluid extraction.

In some embodiments, the present invention utilizes an omega-3 phospholipid composition, preferably a krill oil composition, marine phospholipids form fish roe, fish or fish by-products, or synthetic omega-3 phospholipid, and more preferably a Euphausia superba krill oil composition, comprising from about 40% to about 60% w/w phospholipids, preferably from about 45% to 55% w/w phospholipids. In some embodiments, the composition comprise from about 50 mg/kg astaxanthin to about 2500 mg/kg astaxanthin, preferably from about 1000 to about 2200 mg/kg astaxanthin, more preferably from about 1500 to about 2200 mg/kg astaxanthin. In some preferred embodiments, the compositions comprise greater than about 1000, 1500, 1800, 1900, 2000, or 2100 mg/kg astaxanthin. In some preferred embodiments, the omega-3 phospholipid compositions of the present invention comprise from about 1%, 2%, 3% or 4% to about 8%, 10%, 12% or 15% w/w ether phospholipids or greater than about 4%, 5%, 6%, 7%, 8%, 9% or 10% ether phospholipids. In some embodiments the ether phospholipids are preferably alkylacylphosphatidylcholine, lyso-alkylacylphosphatidylcholine, alkylacylphosphatidyl-ethanolamine or combinations thereof.

In some embodiments, the omega-3 phospholipid compositions comprise from about 1%, 2%, 3% or 4% to about 8%, 10%, 12% or 15% w/w ether phospholipids and from about 30%, 33%, 40%, 42%, 45%, 48%, 50%, 52%, 54%, 55% 56%, 58%to about 60% non-ether phospholipids so that the total amount of phospholipids (both ether and non-ether phospholipids) ranges from about 40% to about 60%. One of skill in the art will recognize that the range of 40% to 60% total phospholipids, as well as the other ranges of ether and non-ether phospholipids, can include other values not specifically listed within the range.

In further embodiments, the compositions comprise from about 20% to 45% w/w triglycerides; and from about 50 to about 2500 mg/kg astaxanthin. In some embodiments, the compositions comprise from about 20% to 35%, preferably from about 25% to 35%, omega-3 fatty acids as a percentage of total fatty acids in the composition, wherein from about 70% to 95%, or preferably from about 80% to 90% of the omega-3 fatty acids are attached to the phospholipids. In some embodiments, the present invention provides encapsulated Euphausia superba krill oil compositions.

The present invention is not limited to the presence of any particular omega-3 fatty acid residues in the omega-3 phospholipid composition. In some preferred embodiments, the omega-3 phospholipid comprises EPA and DHA residues. In some embodiments, the omega-3 phospholipid compositions comprise less than about 5%, 4%, 3% or preferably 2% free fatty acids on a weight/weight (w/w) basis. In some embodiments, the omega-3 phospholipid compositions comprise less than about 25%, 20%, 15%, 10% or 5% triglycerides (w/w). In some embodiments, the krill oil compositions comprise greater than about 30%, 40%, 45%, 50%, 55%, 60%, or 65% phosphatidyl choline (w/w). In some embodiments, the omega-3 phospholipid compositions comprise greater than about 100, 200, 300, 400, or 500 mg/kg astaxanthin esters and up to about 700 mg/kg astaxanthin esters. In some embodiments, the present invention provides omega-3 phospholipid compositions comprising at least 500, 1000, 1500, 2000, 2100, or 2200 mg/kg astaxanthin esters and at least 36% (w/w) omega-3 fatty acids. In some embodiments, the krill oil compositions of the present invention comprise less than about 1.0 g/100 g, 0.5 g/100 g, 0.2 g/100g or 0.1 g/100 g total cholesterol.

In some embodiments, the compositions of this invention (such as those described in the preceding paragraphs) are contained in acceptable excipients and/or carriers for oral consumption. The actual form of the carrier, and thus, the composition itself, is not critical. The carrier may be a liquid, gel, gelcap, capsule, powder, solid tablet (coated or non-coated), tea, or the like. The composition is preferably in the form of a tablet or capsule and most preferably in the form of a soft gel capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In further embodiments, the compositions comprise at least one food flavoring such as acetaldehyde (ethanal), acetoin (acetyl methylcarbinol), anethole (parapropenyl anisole), benzaldehyde (benzoic aldehyde), N butyric acid (butanoic acid), d or l carvone (carvol), cinnamaldehyde (cinnamic aldehyde), citral (2,6 dimethyloctadien 2,6 al 8, gera nial, neral), decanal (N decylaldehyde, capraldehyde, capric aldehyde, caprinaldehyde, aldehyde C 10), ethyl acetate, ethyl butyrate, 3 methyl 3 phenyl glycidic acid ethyl ester (ethyl methyl phenyl glycidate, strawberry aldehyde, C 16 aldehyde), ethyl vanillin, geraniol (3,7 dimethyl 2,6 and 3,6 octadien l ol), geranyl acetate (geraniol acetate), limonene (d, l, and dl), linalool (linalol, 3,7 dimethyl 1,6 octadien 3 ol), linalyl acetate (bergamol), methyl anthranilate (methyl 2 aminobenzoate), piperonal (3,4 methylenedioxy benzaldehyde, heliotropin), vanillin, alfalfa (Medicago sativa L.), allspice (Pimenta officinalis), ambrette seed (Hibiscus abelmoschus), angelic (Angelica archangelica), Angostura (Galipea officinalis), anise (Pimpinella anisum), star anise (Illicium verum), balm (Melissa officinalis), basil (Ocimum basilicum), bay (Laurus nobilis), calendula (Calendula officinalis), (Anthemis nobilis), capsicum (Capsicum frutescens), caraway (Carum carvi), cardamom (Elettaria cardamomum), cassia, (Cinnamomum cassia), cayenne pepper (Capsicum frutescens), Celery seed (Apium graveolens), chervil (Anthriscus cerefolium), chives (Allium schoenoprasum), coriander (Coriandrum sativum), cumin (Cuminum cyminum), elder flowers (Sambucus canadensis), fennel (Foeniculum vulgare), fenugreek (Trigonella foenum graecum), ginger (Zingiber officinale), horehound (Marrubium vulgare), horseradish (Armoracia lapathifolia), hyssop (Hyssopus officinalis), lavender (Lavandula officinalis), mace (Myristica fragrans), marjoram (Majorana hortensis), mustard (Brassica nigra, Brassica juncea, Brassica hirta), nutmeg (Myristica fragrans), paprika (Capsicum annuum), black pepper (Piper nigrum), peppermint (Mentha piperita), poppy seed (Papayer somniferum), rosemary (Rosmarinus officinalis), saffron (Crocus sativus), sage (Salvia officinalis), savory (Satureia hortensis, Satureia montana), sesame (Sesamum indicum), spearmint (Mentha spicata), tarragon (Artemisia dracunculus), thyme (Thymus vulgaris, Thymus serpyllum), turmeric (Curcuma longa), vanilla (Vanilla planifolia), zedoary (Curcuma zedoaria), sucrose, glucose, saccharin, sorbitol, mannitol, aspartame. Other suitable flavoring are disclosed in such references as Remington's Pharmaceutical Sciences, 18th Edition, Mack Publishing, p. 1288-1300 (1990), and Furia and Pellanca, Fenaroli's Handbook of Flavor Ingredients, The Chemical Rubber Company, Cleveland, Ohio, (1971), known to those skilled in the art.

In other embodiments, the compositions comprise at least one synthetic or natural food coloring (e.g., annatto extract, astaxanthin, beet powder, ultramarine blue, canthaxanthin, caramel, carotenal, beta carotene, carmine, toasted cottonseed flour, ferrous gluconate, ferrous lactate, grape color extract, grape skin extract, iron oxide, fruit juice, vegetable juice, dried algae meal, tagetes meal, carrot oil, corn endosperm oil, paprika, paprika oleoresin, riboflavin, saffron, tumeric, tumeric and oleoresin).

In still further embodiments, the compositions comprise at least one phytonutrient (e.g., soy isoflavonoids, oligomeric proanthcyanidins, indol 3 carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, cathechins, gallates, and quercitin). Sources of plant phytonutrients include, but are not limited to, soy lecithin, soy isoflavones, brown rice germ, royal jelly, bee propolis, acerola berry juice powder, Japanese green tea, grape seed extract, grape skin extract, carrot juice, bilberry, flaxseed meal, bee pollen, ginkgo biloba, primrose (evening primrose oil), red clover, burdock root, dandelion, parsley, rose hips, milk thistle, ginger, Siberian ginseng, rosemary, curcumin, garlic, lycopene, grapefruit seed extract, spinach, and broccoli.

In still other embodiments, the compositions comprise at least one vitamin (e.g., vitamin A, thiamin (B 1), riboflavin (B2), pyridoxine (B6), cyanocobalamin (B 12), biotin, ascorbic acid (vitamin C), retinoic acid (vitamin D), vitamin E, folic acid and other folates, vitamin K, niacin, and pantothenic acid). In some embodiments, the particles comprise at least one mineral (e.g., sodium, potassium, magnesium, calcium, phosphorus, chlorine, iron, zinc, manganese, flourine, copper, molybdenum, chromium, selenium, and iodine). In some particularly preferred embodiments, a dosage of a plurality of particles includes vitamins or minerals in the range of the recommended daily allowance (RDA) as specified by the United States Department of Agriculture. In still other embodiments, the particles comprise an amino acid supplement formula in which at least one amino acid is included (e.g., 1-carnitine or tryptophan).

### Experimental

### EXAMPLE 1

### Methodology:

This study comprises a single-center, open-label, randomised multi-dose, two-way crossover study. The two test products were A: Superba™ krill oil (2 g capsule), B: Omega-3 enriched fish oil (2 g capsule).

A total of 28 healthy male and female subjects (14 males, 14 females), aged between 25 and 45 years, took part in this study. Subjects were randomised by sex (1:1) to one of the treatment sequences AB or BA. 20 subjects were considered sufficient to achieve an adequate power to detect differences between the treatments. Therefore, it was not foreseen to replace drop-outs after the first dose.

In each treatment period, subjects were confined in the clinic from the evening of Day −2 to the morning of Day 2 (i.e. from 36 h prior to the first dose to 24 h after the first dose of the respective period). Subsequent dosing for the rest of the period was done at home. During treatment periods, clinic visits were scheduled every 2 weeks (±2 days). On Day 2 and every two weeks (±2 days), subjects were provided with product supply for home consumption in the next two weeks (plus spare supplies). Between clinic visits, subjects were contacted per telephone every 2 weeks (±2 days) to monitor safety. These phone calls included an inquiry for adverse events and concomitant medication and ensured treatment compliance.

One dose was taken daily at the same time of the day, preferably in the morning after breakfast. There was a 8-week wash out phase between the 2 treatment periods.

Except on days of confinement, subjects filled out a diary to document daily food consumption (e.g., fatty fish or seafood, cholesterol-lowering products, or omega-3 enriched products) during the entire study, including the wash-out phase. A list of food to be avoided was provided. During the treatment periods, the diary was also used to document daily dosing. The volunteers began to keep this diary in the morning of Day 2 (Period 1) by entering their dose for this day.

Number of subjects:

Planned sample size: N=28 healthy, adult (14 Males, 14 females)

Actual sample size: N=28 healthy, adult (14 Males, 14 females)

PP population: N=26 healthy, adult (13 Males, 13 females)

Safety population N=28 healthy, adult (14 Males, 14 females)

### Main Criteria for Inclusion:

Healthy non-smoking male and female subjects, aged between 25 and 45 years. Subjects with cardiovascular diseases or allergies against crustacean were excluded from participation in the study.

Restrictions:
No fatty fish or seafood meals 3 days before dosing and during treatment periods
Fatty fish meals not more than once per week during wash-out phase
Functional food like cholesterol-reducing products, lipid supplements, and omega-3 fatty acids containing food is not allowed during the entire study
Lipid-lowering medicine is not allowed during the entire study.

### Criteria for Evaluation:

Blood for the pharmacokinetic analysis was collected on Day 1 (pre-dose) and on Days 14, 28, 42 and 56 (±2 days) of each treatment period for the analysis of EPA and DHA in phospholipid fractions and of omega-3 index in RBCs.

Pharmacokinetic evaluation: Primary parameter: AUC_{(0-50D)} AUC_{(0-56D)}: The primary pharmacokinetic variables were the areas under the concentration vs. time curves of EPA, DHA and the omega-3 index from Day 0 to Day 56. AUC_{(0-56D)} was determined after baseline correction of the concentration values after dose-adjustment for EPA and DHA and the omega-3 index.

Pharmacodynamic evaluation: Blood for pharmacodynamic evaluation was collected on Day 1 (pre-dose) and on Day 56 (±2 days) of each period for the following:
Platelet aggregation tests (ADP test, ASPI test, TRAP test).
Lipid profile (total cholesterol, HDL, LDL, and triglycerides)
Reduced clinical chemistry (glucose, CRP, insulin, TNF-alpha, and adiponectin)
   Primary parameter: the changes from baseline of platelet aggregation tests (ADP test, ASPI test, TRAP test), lipid profile (total cholesterol, HDL, LDL, and triglycerides) and reduced clinical chemistry (glucose, CRP, insulin, TNF-alpha, and adiponectin). Descriptive statistics were provided by treatment for the variables as measured at baseline and at Day 56, and for the changes from baseline.

### Safety:

Safety assessments included the inquiry about adverse events and concomitant medication at all study days, as well as a physical examination at Screening and on Day 56 of Period 2, the recording of adverse events at each clinic visit and at the phone calls, the measurement of vital signs (blood pressure, pulse rate, body temperature), the recording of a 12-lead ECG, and a standard clinical laboratory assessment (urinalysis, haematology, clinical chemistry) at Screening and on Day 56 of Period 2.

### Other Assessments:

Additional assessments included demographic data, medical history at Screening, smoking, and caffeine status, and diet at Screening, serology at Screening, urine drug screen and alcohol breath test, pregnancy test performed on female subjects at Screening and on Day −2 of both periods.

### Statistical Methods:

Statistical Analysis of Pharmacokinetic Parameters

The primary statistical analysis was performed on the baseline corrected area under the data points AUC_{(0-56D)} from week 0 to week 8 of the omega-3 index in RBCs as well as on baseline corrected total plasma fatty acids (including EPA and DHA). A multiplicative model was used. The AUC_{(0-56D)} values were log-transformed and subjected to an ANOVA model including the factors “product”, “period”, “sequence”, and “subjects within sequence”. The ANOVA was used to obtain point- and interval estimates for the difference between the test products in the log-AUC_{(0-56D)} values, which after back-transformation corresponded to the geometric mean of the individual ratios (A/B) and its 90% confidence interval (CI). Superiority of A in comparison to B was accepted if the lower limit of the 95% CI of the ratio is greater than 1.0 (i.e. the entire CI lies above 1.0). This data analysis approach corresponded to a one-sided t-test at the 2.5% level of significance.

Steady State Analysis: Attainment of steady state was checked by a repeated measurement ANOVA followed by comparisons of each day with the mean of the subsequent days (Helmert transformation). The ANOVA model included the terms “subject” and “day” and was determined for each test product separately. Attainment of steady state was assumed when a contrast (and all subsequent contrasts) showed no significant difference (α=0.05, two-sided). Additionally, the products were compared regarding the EPA, DHA and omega-3 index curves (baseline corrected and dose-adjusted) at each time point using the non-parametric Wilcoxon signed rank test.

Statistical Analysis of Pharmacodynamic Parameters

Secondary statistical analyses: Changes of Lipid profile (total cholesterol, HDL, LDL, and triglycerides) and changes of the reduced clinical chemistry panel (glucose, CRP, insulin, TNF-alpha, and adiponectin) from baseline to Day 56 were analysed in the framework of a repeated measurement ANOVA model.

A similar to the above ANOVA model for AUC(0-56D) values were used to analyse changes in platelet aggregation from baseline to Day 56. Differences in changes from baseline to Day 56 between Superba™ krill oil and omega-3 enriched fish oil (α=0.05, two-sided) regarding PD variables were analysed by using an ANOVA model including the factors “product”, “period”, “sequence”, and “subjects within sequence”.

### Results:

Pharmacokinetics: Quantifiable concentrations of EPA and DHA in plasma and of omega-3 fatty acids in RBCs were observed in all subjects after multiple doses of Superba™ krill oil and omega-3 enriched fish oil. Generally, there was a steep increase in the levels of EPA and DHA in plasma and omega-3 index in RBCs from baseline to Day 14 after both products. Steady state in EPA levels and omega-3 index was attained earlier after Superba™ krill oil (Day 14) as compared to omega-3 enriched fish oil (Day 28). Steady state in DHA levels was attained later after Superba™ krill oil (Day 42) than after omega-3 enriched fish oil (Day 28).

Non-parametric comparison of the products regarding the EPA, DHA and omega 3 index curves (baseline corrected and dose-adjusted) at each time point showed statistical significance on Day 14 (p=0.007), Day 42 (p=0.041), and Day 56 (p=0.027) for EPA concentrations in plasma. Statistical significance was reached on Day 42 (p=0.041), and Day 56 (p=0.016) for DHA concentrations in plasma. No statistical significance could be demonstrated for omega-3 index in RBCs.

| | Product | | | | | | |
|---|---|---|---|---|---|---|---|
| | Superba ™ krill oil | | | Omega-3 enriched fish oil | | | |
| Variable | | Arithmetic | | | Arithmetic | | ANOVA |
| (AUC_{(0 56D)}) | N | Mean | SD | N | Mean | SD | p-value |
| EPA [ng*h/(mg*ml)] | | | | | | | |
| Overall | 25 | 97908.4 | 47899.9 | 28 | 81312.4 | 30740.7 | 0.778^{a} |
| Males | 12 | 79009.7 | 48381.2 | 11 | 75014.4 | 30330.9 | |
| Females | 13 | 115353.6 | 41960.7 | 12 | 67087.7 | 31266.9 | 0.026^{b} |
| DHA [ng*h/(mg*ml)] | | | | | | | |
| Overall | 25 | 98261.2 | 52402.0 | 23 | 78943.4 | 41463.5 | 0.027^{b} |
| Males | 12 | 81956.7 | 51443.0 | 11 | 73845.3 | 38956.8 | |
| Females | 13 | 113302.0 | 5062.0 | 12 | 79588.9 | 45579.1 | 0.182^{b} |
| Omega-3 index in RBCs [%*h/g] | | | | | | | |
| Overall | 23 | 4207.8 | 3653.3 | 23 | 2499.1 | 1931.3 | 0.152^{a} |
| Males | 12 | 3627.8 | 3606.1 | 12 | 2630.4 | 2219.0 | |
| Females | 11 | 4640.5 | 3780.0 | 11 | 2348.0 | 1655.4 | 0.356^{b} |

All AUC-values are baseline corrected and dose adjusted
ANOVA calculated after logarithmic transformation:
^{a}p-calue for treatment:
^{b}p-value sex treatment interaction
Subject 112 was excluded from the analysis of EPA.
The unexpected low omega-3 index values were esculated from analysis.

Superiority of Superba™ krill oil vs. omega-3 enriched fish oil could be demonstrated in female subjects with respect to the bioavailability of EPA in plasma (after dose adjustment) and across males and females with respect to DHA in plasma (after dose adjustment). Statistically significant differences between the treatments could not be demonstrated with respect to omega-3 index in RBCs (after dose adjustment). Exclusion of invalid omega-3 index values at discrete time points and the subsequent loss of statistical power should be taken into account on considering this result.

There was an unexpected general tendency to higher concentration levels in EPA, DHA and omega-3 index for females after both products and a tendency to larger treatment differences in females as compared to males. See FIGS. 1-5.

Pharmacodynamics: After Superba™ krill oil, the mean serum insulin level decreased, whereas the mean adiponectin level increased. On the other hand, after omega-3 enriched fish oil, both the mean serum insulin level and the mean adiponectin level decreased. Otherwise, there were no relevant changes after both treatments, and no statistically significant differences between Superba™ krill oil and omega-3 enriched fish oil in any of the parameters analysed, including platelet aggregation tests (ADP, ASPI, and TRAP tests) lipid parameters (triglycerides, LDL, HDL and total cholesterol) and other selected clinical chemistry parameters (glucose, CRP, insulin TNF alpha, and adiponectin).

### EXAMPLE 2

The objective of this study is to compare the gastrointestinal tolerability of Superba krill oil to fish oil in a survey of a random group of healthy men and women accessed through physicians' offices.

Subjects will report to the clinic to be certain they qualify for the study, be given instructions for dosing and receive survey product. Subjects will be asked to consume 2 gm daily of either Superba or a commonly consumed brand (Brand XX) of fish oil for two (2) weeks. Trial preparations (4 capsules of 500 mg fish oil and 4 capsules of 500 mg krill oil) should be taken all together as a single dose with about 8 ounces of water 2-3 hours after the breakfast meal. No other food or drink should be taken during the 2 hours before and after the dose.

Subjects will return to the clinic at the end of the two week dosing period to complete visual analog scales (VASs) for taste, odor, eructation (burping) and overall tolerability. After a 3 week washout, subjects will return to the clinic to receive a two (2) week supply of the product not taken in the first session and will return two weeks later to complete VASs, as before. In addition, subjects will be asked which of the products they preferred.

Products will be repackaged in identical appearing bottles identified only by subject initials and number and product ‘A’ or ‘B’. Although the capsules will not be identical, they will bear no identifying marks, thus allowing the survey to be conducted in a double blind fashion. The order in which subjects will be allocated survey product will be randomly generated by computer.

Inclusion Criteria:
1-either gender, age 40-75 years
2-in general good health
3-has not taken any O-3 product for at least 3 months prior to beginning this survey
4-agrees to refrain from using any O-3 product for the duration of the survey

Exclusion Criteria:
1-prior history of severe intolerance to any O-3 product
2-Allergy to seafood products or iodine
3-history of any gastrointestinal disease that might interfere with absorption of
O-3 oils including, but not limited to, chronic ulcer disease, pancreatitis, biliary disease, inflammatory bowel disease or chronic diarrhea

Statistical analysis: Mean VAS scores for each parameter will be compared between groups by Chi Square analysis. In addition, a secondary analysis will be performed to examine the effect of the order in which products were taken. Data on overall product preference will be presented as a percent preference, if any, of one product versus the other.

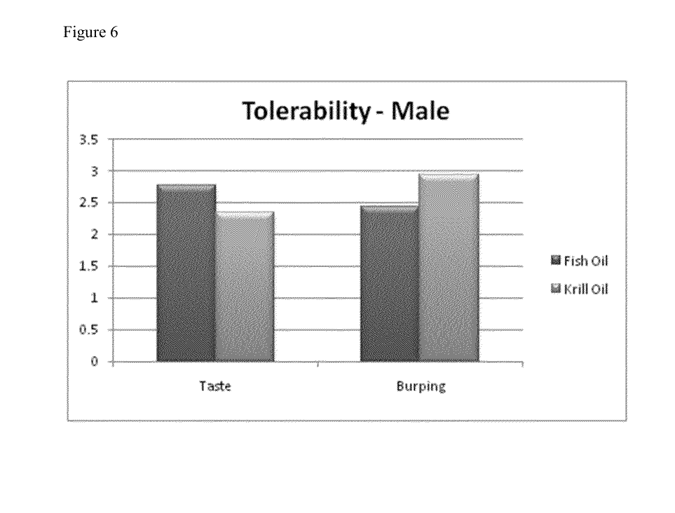

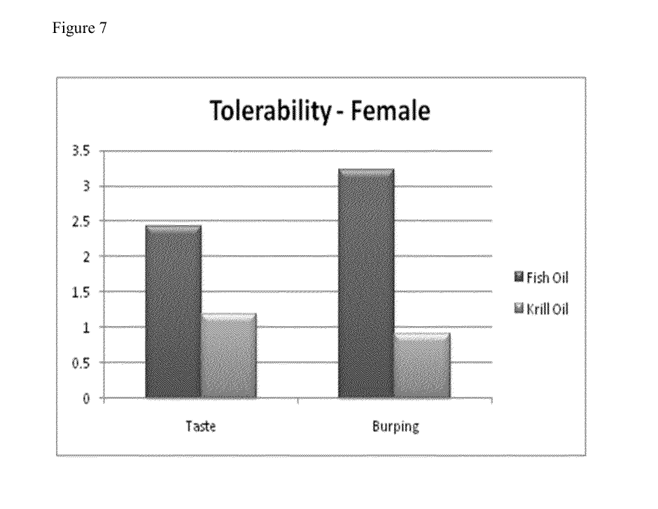

The results from the study are presented in FIGS. 6 and 7. As can be seen, there were distinct differences in tolerability between the genders, with females showing distinct differences in reduction of burping and taste response.

### EXAMPLE 3

A double-blind, randomized, placebo controlled, study was conducted to investigate the effects of 4 gram krill oil on the development of the omega-3 index in male and female subjects. The omega-3 index is defined as the percentage of EPA and DHA in red blood cell fatty acids and it has been proposed as a novel biomarker for cardiovascular risk.

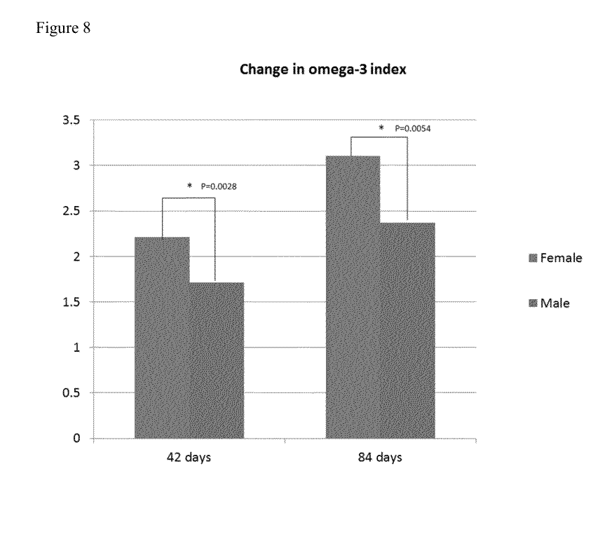

The subjects received 4 g krill oil daily for 12 weeks. The results are shown in FIG. 8 and demonstrate a 29% and 31% higher increase in omega-3 index in female versus male, after 6 and 12 weeks of krill oil intake, respectively.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method of increasing the omega-3 phospholipid content of plasma phospholipids in a female subject as compared to male subjects comprising:
administering an omega-3 phospholipid supplement to said female subject under conditions such that the omega-3 phospholipid content of plasma phospholipids in said female subject is increased.

2. The method of claim 1, further comprising administering to said female subject from about 2 to 6 grams of said omega-3 phospholipid supplement for a period at least six weeks to effect an increase in omega-3 index of from about 1.8 to 2.5 fold as compared to control subjects not receiving the treatment.

3. The method of claim 1, further comprising administering to said female subject from about 2 to 6 grams of said omega-3 phospholipid supplement for a period at least twelve weeks to effect an increase in omega-3 index of from about 2.5 to 3.5 fold as compared to control subjects not receiving the treatment.

4. The method of claim 1, wherein said female subject is not receiving a concurrent lipid altering therapy.

5. The method of claim 1, wherein said omega-3 phospholipid supplement is a krill oil, fish oil, fish roe oil, or fish byproduct oil.

6. The method of claim 5, wherein said krill oil comprises from about 35% to 60%; from about 20% to 45% triglycerides on a w/w basis; and from about 50 to about 2500 mg/kg astaxanthin.

7. The method of claim 6, wherein said composition comprises from about 3% to 10% ether phospholipids on a w/w basis, so that the total amount of ether phospholipids and non-ether phospholipids in the composition is from about 48% to 60% on a w/w basis.

8. The method of claim 7, wherein said composition comprises from about 25% to 30% omega-3 fatty acids as a percentage of total fatty acids and wherein from about 80% to 90% of said omega-3 fatty acids are attached to said phospholipids.

9. The method of claim 8, wherein said composition comprises from about 100 to about 2500 mg/kg astaxanthin.

10. The method of claim 1, wherein said omega-3 supplement comprises from about 1% to about 10% w/w ether phospholipids; from about 27% to 50% w/w non-ether phospholipids so that the amount of total phospholipids in the composition is from about 30% to 60% w/w; from about 20% to 50% w/w triglycerides; from about 100 to about 2500 mg/kg astaxanthin; and from about 20% to 35% omega-3 fatty acids as a percentage of total fatty acids in said composition, wherein from about 70% to 95% of said omega-3 fatty acids are attached to said phospholipids.

11. The method of claim 1, wherein said omega-3 is selected from EPA and DHA and combinations thereof.

12. The method of claim 1, wherein said female subject is a human.

13. The method of claim 1, wherein said administration is oral.

14. The method of claim 1, wherein the omega-3 index is increased in said female subject as compared to a male subject.

15. The method of claim 1, wherein said administration effects a 35% to 55% increase in EPA in plasma phospholipids as compared to males and a 30% to 50% increase in DHA in plasma phospholipids as compared to males.

16. The method of claim 1, wherein said administration effects a 20% to 40% increase in EPA in plasma phospholipids as compared to females receiving fish oil and a 30% to 50% increase in DHA in plasma phospholipids as compared to females receiving fish oil.

17. The method of claim 1, wherein said administration of said omega-3 supplements effects a reduction in burping as compared to control subjects receiving fish oil.

18. A method of increasing the omega-3 index in a female subject as compared to male subjects comprising:
administering to said female subject from about 2 to 6 grams of an omega-3 phospholipid supplement for a period at least twelve weeks to effect an increase in omega-3 index of from about 2.5 to 3.5 fold as compared to control subjects not receiving the treatment.

19. A method of increasing the omega-3 index in a female subject as compared to male subjects comprising:
administering to said female subject from about 2 to 6 grams of a krill oil supplement for a period at least twelve weeks to effect an increase in omega-3 index of from about 2.5 to 3.5 fold as compared to control subjects not receiving the treatment, wherein said krill oil supplement comprises from about 1% to about 10% w/w ether phospholipids; from about 27% to 50% w/w non-ether phospholipids so that the amount of total phospholipids in the composition is from about 30% to 60% w/w; from about 20% to 50% w/w triglycerides; from about 100 to about 2500 mg/kg astaxanthin; and from about 20% to 35% omega-3 fatty acids as a percentage of total fatty acids in said composition, wherein from about 70% to 95% of said omega-3 fatty acids are attached to said phospholipids.
